(19) United States
(12) Patent Application Publication
(10) Pub. No.: US 2022/0313887 A1
(43) Pub. Date: Oct. 6, 2022

(54) PRESSURE-MEASURING ASSEMBLY FOR AN EXTRACORPOREAL BLOOD TREATMENT MACHINE

(71) Applicant: B. Braun Avitum AG, Melsungen (DE)

(72) Inventor: Kai-Uwe Ritter, Melsungen (DE)

(21) Appl. No.: 17/615,924

(22) PCT Filed: Jun. 4, 2020
(86) PCT No.: PCT/EP2020/065477

§ 371 Date: Dec. 2, 2021

(30) Foreign Application Priority Data
Jun. 6, 2019 (DE) 10 2019 115 271.2

(51) Int. Cl. A61M 1/36; A61M 39/12; A61M 39/10

(52) U.S. Cl. CPC A61M 1/3639; A61M 39/12; A61M 39/1011; A61M 2205/3331

(57) ABSTRACT

A pressure-measuring assembly for measuring the internal line pressure of a line system of an extracorporeal blood treatment machine. The assembly includes a pressure sensor that is connectable to a pressure receiver by a rigid, bending-resistant fluid line. The pressure receiver is connected or connectable to the line system. The pressure sensor converts a fluid pressure signal from the fluid line into an electrical signal. An electrical line connects the pressure sensor to electronics to process the electrical signal. A retainer directly or indirectly retains the pressure sensor and forms, at least in parts, an inner channel, in which the electrical line is led. Alternatively, the retainer is a substantially plastically curvable bar which has, at one axial end portion, a fastening device for mounting on a stationary base and, at the other axial end portion, an articulation site for the pressure sensor or the pressure receiver.

### CROSS-REFERENCE TO RELATED APPLICATIONS

This application is the United States national phase entry of International Application No. PCT/EP2020/065477, filed Jun. 4, 2020, and claims priority to German Application No. 10 2019 115 271.2, filed Jun. 6, 2019. The contents of International Application No. PCT/EP2020/065477 and German Application No. 10 2019 115 271.2 are incorporated by reference herein in their entireties.

### FIELD

The present invention relates to a pressure-measuring assembly for measuring the internal line pressure of a preferably extracorporeal line system of an extracorporeal blood treatment machine, in particular a dialysis machine, comprising a pressure sensor which can be connected via a rigid, bending resistant fluid line, preferably a gas line, to a pressure receiver connected or connectable to the line system and which converts a fluid pressure signal from the rigid, bending resistant fluid line into an electrical signal, an electric line, preferably a cable, which serves to connect the pressure sensor to electronics for processing the electrical signal, and a retainer for directly or indirectly holding the pressure sensor. Furthermore, the present invention relates to an extracorporeal blood treatment machine with a corresponding pressure-measuring assembly.

### BACKGROUND

Extracorporeal blood treatment machines, in particular dialysis machines, have a line system, in particular with a dialysis fluid circuit as well as an extracorporeal blood circuit. These circuits have a number of dialysis fluid lines and blood (tubing) lines that pass through and/or connect various functional units of the extracorporeal blood treatment machine, such as a dialyzer, blood pumps, etc. In order to ensure smooth functionality of the extracorporeal blood treatment machine, this line system, in particular the blood tube lines, have to be pressure monitored at several locations. For example, in a common dialysis machine, a port (“PA port”) is provided to monitor the arterial negative pressure, a pressure-before-entry port (“PBE port”) is provided to monitor the inlet pressure before the dialyzer, and a port (“PV port”) is provided to monitor the venous pressure.

For the purpose of pressure monitoring, branches or so-called pressure oscillating diaphragms (“PODs”) are installed as pressure receivers at suitable locations in the lines or blood tube lines, which are also referred to below as pressure-receiver capsules. Corresponding PODs or pressure-receiver capsules are known from the prior art. For example, U.S. Pat. No. 8,092,414 B2 and U.S. Pat. No. 8,491,518 B2 disclose pressure-receiver capsules with a volume-rigid capsule, which is divided by a diaphragm into two chambers, more precisely into a first chamber connected to a line system and into a second (air-side) chamber, which is connected via a thin tube to a pressure sensor or an associated port.

In other words, the branches or PODs are connected or attached to the machine (extracorporeal blood treatment machine), more specifically, to a housing of the machine, via thin tubes at pressure receiver ports (pressure transducer ports). The ports are typically equipped with Luer lock connectors. Since sensors are often used which are not suitable to be led directly through a housing to the outside, an additional tube inside the housing may be necessary in order to connect the pressure receiver port of the housing with the pressure sensor located inside the housing. Alternatively, pressure sensors are known which are provided directly with a Luer lock/slip connector via a thin tube.

The fact that the pressure-receiver capsules are suspended in the tube lines of the line system and are therefore in not precisely defined, unstable positions can lead to measurement inaccuracies. It is known from U.S. Pat. No. 9,393,397 B2 that this problem can be avoided by attaching the pressure-receiver capsule directly to a dialyzer. However, problems caused by the thin tube itself cannot be avoided by this. That means that this still has the disadvantage of the long tubing connection between the POD (pressure receiver) and pressure receiver port (pressure transducer port). For example, the thin tube is susceptible to damage such as kinking by a careless user. Furthermore, a large number of transitions or connection sites are necessary, in particular if an additional tube inside the housing is provided, which increases the risk of leaks in the line and also results in a complex, expensive assembly. Furthermore, it is easy for a user to accidentally connect the tube, which is to be attached to a pressure receiver port of the housing via a standardized interface such as a Luer lock connector, to an incorrect port. Such an incorrect connection may lead to interruptions or delays of a therapy, in particular a dialysis therapy, and in the worst case this may endanger patients or staff.

During operation, an internal line pressure present in the line system and thus in the first chamber is transmitted via the diaphragm to the second chamber, in which a fluid pressure, in particular a gas pressure, is generated that is dependent on the internal line pressure. The pressure sensor connected to the second chamber via the thin tube thus receives a fluid pressure signal, which it converts into an electrical signal. Due to the thin, flexible tube, which forms a large fluid or air path and a dead volume between the pressure-receiver capsule and the pressure sensor, a low-pass effect is created, so that the accuracy of the measurement and a response time of the pressure sensor is limited.

Pressure-receiver capsules or PODs are known from U.S. Pat. No. 8,210,049 B2 and EP 1 843 140 A2, in which the pressure-receiver capsule is connected to the pressure sensor directly or via a short, volume-rigid connection in order to improve measurement accuracy. The pressure sensor may be attached to a frame of a permanent retainer. If such a retainer is provided, for example, directly in a housing of an extracorporeal blood treatment machine, assembly of the line system may be cumbersome and complex. In particular, it may be difficult or even impossible to use different line systems or blood tube lines. This means that the retainer is presumably not configured or sufficiently flexible to allow a number of different line systems to be connectable to it. If the permanent retainer is provided outside the housing, e.g. on a dialyzer or on a blood pump, an electric line has to be provided between the retainer and signal processing electronics, which may easily be damaged by environmental influences such as careless users, aggressive disinfectants, etc., and a position of the pressure sensor is selectable only to a limited extent.

### SUMMARY

The object underlying the present invention is to improve or eliminate disadvantages of the prior art. In particular, a pressure-measuring assembly for an extracorporeal blood treatment machine is to be provided that is configured/suitable for use with differently configured line systems, that makes it impossible to incorrectly connect pressure receivers of the line system, and that enables a functionally reliable and precise measurement of an internal line pressure.

The core idea of the invention is to provide a pressure-measuring assembly for measuring an internal line pressure of a line/line system of an extracorporeal blood treatment machine, which provides a retainer external to the machine, preferably 15 to 30 cm long, for holding a pressure sensor and a pressure receiver of the line/line system, which are connectable to each other via a rigid, in particular volume-constant fluid line. The retainer is configured to hold the pressure sensor directly or indirectly (e.g. via the pressure receiver) in such a way that an electrical connection is protected from environmental influences by the retainer itself and/or that a position of the pressure sensor is adjusted or adjustable in such a way that different lines/line systems are easily connectable or usable with the provided pressure-measuring assembly.

More precisely, the object underlying the invention is solved by a pressure-measuring assembly for measuring the internal line pressure of a preferably extracorporeal line system (at a certain location/line portion thereof) of an extracorporeal blood treatment machine, in particular a dialysis machine, with a pressure sensor which is connectable via a rigid, (plastically and substantially also elastically) bending resistant (in particular volume-constant) fluid line, preferably a gas line, to a pressure receiver connected or connectable to the line system and converts a fluid pressure signal from the rigid, bending resistant fluid line into an electrical signal, with an electrical line, preferably a cable, which serves to connect the pressure sensor to electronics in order to process the electrical signal, and with a retainer for directly or indirectly holding the pressure sensor.

The retainer of the aforementioned pressure-measuring assembly is formed as a substantially plastically bendable (in particular by a user) rod having, at one axial end portion, a fastening device for being mounted to a stationary base and, at the other axial end portion, an articulation site for the pressure sensor or pressure receiver. In other words, the retainer has a longitudinal extension that allows the pressure sensor to be held in an optimally selected or adjustable position outside the housing of the extracorporeal blood treatment machine. Preferably, the rod is configured to be flexurally rigid, which in this context means that the retainer/rod does not bend or bends only minimally elastically and non-plastically relative to the inherent weight of the pressure sensor and of the pressure receiver connected thereto and, if applicable, of the lines, and preferably under an application-related vibration, in order to provide fixed positioning of the pressure sensor in space. On the other hand, as described above, the rod is plastically deformable under a higher force application that is possible in particular for a user. Alternatively or in addition to the configuration as a (flexurally rigid) plastically bendable rod, as described above, the retainer forms, at least in sections, an inner channel in which the electric line is guided such that it is protected from the environment. I.e., a wall of the inner channel or a structure or material of the retainer is selected in such a way that it is more stable, in particular harder and/or more bending resistant than the electric line (i.e., a greater force is required to bend the retainer than the electric line) and preferably resistant to environmental influences such as aggressive disinfectants.

In the aforementioned pressure-measuring assembly, no additional thin tube is required to connect the pressure receiver, which saves costs since such a tube is usually provided as a disposable part. Furthermore, a number of interfaces that may be subject to leakage is thus small and incorrect connection of the thin tube is excluded. In addition, it is advantageous that the dead volume of the otherwise existing tubes can be reduced and a low-pass effect, which is caused by the air gap in such a tube, can be prevented. In particular, it is advantageous if the rigid, bending resistant fluid line between the pressure receiver and the pressure sensor is kept as short as possible. Accordingly, it is possible to ensure more accurate pressure measurement and shorter reaction times of the pressure sensor to pressure changes of the line system, in particular in a blood tube line of the extracorporeal blood treatment machine. It is furthermore advantageous that the pressure sensor can be held outside the housing in a suitable, preferably adjustable position and/or that damage to the electrical line can be avoided due to the protective inner channel.

A coupling portion, preferably a Luer-lock/slip connector or a click-connector/latchable connector, is provided for connecting the pressure sensor to the pressure receiver via the rigid, bending resistant fluid line. These ports are particularly simple, inexpensive, and easy to use. Furthermore, the rigid, bending resistant fluid line is preferably formed by the coupling portion to keep an internal volume of the rigid, bending resistant fluid line (its length) as small (short) as possible. This further increases the measurement accuracy.

In order to protect the electric line particularly effectively, it is advantageous if the inner channel forms a receiving space for the electric line that is completely closed with respect to the outside.

The retainer is capable of holding the pressure sensor directly, which makes it possible to connect the pressure receiver of the line system to the pressure sensor using a single interface when preparing an extracorporeal blood treatment. This ensures a particularly simple and fast assembly of the line system. Alternatively, the retainer may hold the pressure sensor indirectly, for example via the pressure receiver. I.e., in order to mount the line system, the pressure receiver is first attached to the retainer and then the pressure sensor is connected to the pressure receiver via the rigid, bending resistant fluid line. It is conceivable to configure the retainer for this purpose in such a way that an electrical connection is created when the pressure sensor is connected, for example via a touch contact, induction or a plug-in connection.

Preferably, the retainer is plastically deformable or curvable, further preferably in the form of a gooseneck, in such a way that a position of the articulation site is preferably manually adjustable. This allows the pressure sensor held at the articulation site to be moved out of the way during preparation of the dialysis treatment or to be deformed accordingly and to be adjusted to a position suitable for connecting the pressure receiver during assembly of the line system. This means that the retainer can be easily adapted to differently configured line systems during installation of the line system. It can also be said that the retainer according to this embodiment is a semi-rigid, bendable connecting element, for example made of a coiled material (e.g. metal tube), which can be bent in almost any direction and can remain in any position. In particular, it is advantageous if the inner channel is formed by the gooseneck. In this way, it is possible to adjust/set the pressure sensor via the gooseneck without damaging the electric line and also to protect it against environmental influences.

Alternatively, the retainer may be a rigid, preferably hollow rod extending in a predetermined linear or curved shape. According to this embodiment, the retainer may be shaped such that the articulation site is located at a position suitable for connecting the line system and the risk of inaccurate measurement due to the position of the pressure sensor changing during dialysis treatment (after calibration of the pressure sensor) can be substantially completely prevented.

It is advantageous if the pressure receiver is configured in the manner of a pressure-receiver capsule, which comprises a first chamber connected or connectable to the line/line system and a second, preferably gas-filled chamber, which is separated from the first chamber by a diaphragm and to which the pressure sensor is coupled or couplable. In other words, the pressure receiver is designed as a pressure-receiver capsule which allows to reliably measure an internal line pressure without compromising the sterility of, for example, a blood line to be measured. Preferably, in this case, a valve integrated or mounted on the pressure sensor or on the rigid, bending resistant fluid line is provided in order to vent the pressure-receiver capsule, in particular the second chamber. This means that the valve is fluidically connected to the second chamber of the pressure-receiver capsule or to the rigid, bending resistant fluid line. This makes it possible to bring the diaphragm of the pressure-receiver capsule into a certain position by opening the valve and orienting the diaphragm by changing the pressure, e.g. on the blood side. The valve is then hermetically closed again. This means that the valve is associated with the pressure sensor and is thus provided as a reusable part. The pressure-receiver capsule, which is usually provided as a disposable part, therefore does not require a valve and can be manufactured at lower cost.

Preferably, the retainer holds the pressure sensor, the pressure sensor holds the rigid, bending resistant fluid line (or is attached thereto, in particular via the coupling portion), and the fluid line is adapted to be attached to the pressure receiver. In other words, the retainer, the pressure sensor and the rigid, bending resistant fluid line and, if applicable, the pressure receiver are arranged or connected to each other in this order. In particular, these elements are part of the following signal transmission device. When a pressure receiver is connected to the rigid, bending resistant fluid line, a pressure signal (in particular a gas/air pressure) is transmitted to the pressure sensor via the rigid, bending resistant fluid line. Preferably, the fluid line is arranged (directly) on the pressure sensor (connects the pressure receiver and the pressure sensor) and is continuously rigid and bending resistant. The pressure sensor converts the pressure signal into an electrical signal. From the pressure sensor, the electrical signal is transmitted to an electric line, which runs through the inner channel of the retainer and is configured to transmit the electrical signal from the pressure sensor to a control unit.

In particular, the control unit is housed (protected) in an interior space of a housing. The retainer, the pressure sensor and the rigid, bending resistant fluid line are in particular arranged on a part of the housing facing outwards. Preferably, the retainer is adapted to be attached (in particular on the outside/facing a housing exterior) to a housing portion of the extracorporeal blood treatment machine. In other words, preferably an end of the retainer opposite to the pressure sensor is attached to a side (facing away from/towards the outside of the housing interior) of the housing portion, in particular a housing plate, and further preferably the control unit is arranged on an opposite side (facing the housing interior) of the housing portion. The electric line runs from the pressure sensor through the retainer to the housing portion, preferably through the housing portion and inside the housing to the control unit. The electric line thus has, in particular, a first (external) portion guided in the retainer and a second (internal) portion arranged on the side of the housing portion opposite the retainer. The first and second portions may be formed separately and may be connected or connectable to each other via a connector of the housing portion.

The object underlying the invention is furthermore solved by an extracorporeal blood treatment machine, in particular a dialysis machine, having a pressure-measuring assembly described above. In particular, the blood treatment machine has a housing portion, in particular with a surface facing the outside of the housing, to which the retainer is attached.

In summary, the object underlying the invention is solved by a retainer attached to the housing of the machine (extracorporeal treatment machine), e.g. in the form of a gooseneck, which is preferably 15 to 30 cm long. A pressure transducer (pressure sensor) is attached to the end of the retainer. The pressure can thus be measured directly at the POD (pressure-receiver capsule). The electrical signals are transmitted via lines in the retainer to a control and monitoring unit (electronics). This has the advantage that the number of interfaces is reduced, that the connecting tube at the POD can be omitted and thus costs can be reduced, that the dead volume in otherwise existing tubes is reduced and that there is no low-pass effect of the air gap in an otherwise existing tube, whereby the sensor has shorter reaction times to pressure changes on the blood side. The POD can be moved to a defined position using the retainer, which is preferably in the form of a gooseneck. This retainer allows the position of the POD to be adapted to different break tube lines.

As an alternative to the POD connection (connection between pressure-receiver capsule and pressure sensor) via Luer, a latching click connection can be used. The seal can then preferably be made radially. A valve can additionally be installed in the pressure sensor or on the rigid, bending resistant fluid line in order to be able to ventilate the POD. This is necessary if the POD diaphragm is to be brought into a certain position. For this purpose, the valve can be opened and the diaphragm can be oriented by changing the pressure on the blood side. After this process, the valve is hermetically closed again. Instead of semi-flexible retainers, e.g. in the form of a gooseneck, a fixed connection that protrudes from the machine can also be provided.

### BRIEF DESCRIPTION OF THE DRAWING FIGURES

The present invention is described below with reference to preferred embodiments.

However, these are only illustrative in nature and are not intended to limit the scope of protection of the present invention. Furthermore, identical reference signs are used for the same components in the description of the various embodiments in order to avoid redundant descriptions of the same.

FIG. 1 shows a pressure-measuring assembly of a first embodiment of the invention with a pressure receiver connected thereto.

FIG. 2 shows a detailed view of a coupling of the pressure receiver with the pressure-measuring assembly of the first embodiment.

FIG. 3 schematically illustrates a pressure-measuring assembly according to a second embodiment of the invention.

FIG. 4 shows further aspects of the pressure-measuring assembly according to the first or second embodiment.

### DETAILED DESCRIPTION

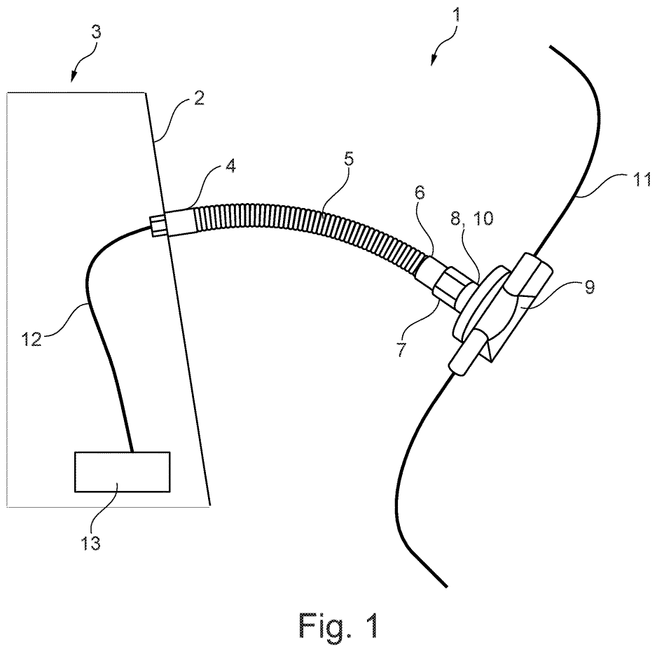

FIG. 1 shows a pressure-measuring assembly 1 according to a first embodiment of the invention. A retainer 5 is attached via a fastening device 4 to a housing 2, which forms the stationary base, of an extracorporeal blood treatment machine 3. The retainer 5 is rod-like or tubular in shape and extends outwards from the housing 2. More specifically, according to this embodiment, the retainer 5 forms a gooseneck. A gooseneck is a semi-rigid, (manually/plastically) flexible or bendable, in particular (elastically) flexurally rigid connecting element, which can be bent manually in almost any direction and can remain in a correspondingly adjusted position. FIG. 1 schematically shows a gooseneck made of a coiled metal tube.

The retainer 5 forms an articulation site 6 on a side facing away from the housing 2 or on an axial end portion opposite the fastening device 4. A pressure sensor 7 is mounted on this articulation site 6. The pressure sensor 7 has a coupling portion 8, through which it is connected to a pressure-receiver capsule 9, which forms a pressure receiver. The coupling portion 8 further forms a very short, rigid, bending resistant fluid line 10 which connects the pressure sensor 7 to the pressure-receiver capsule 9, more specifically to an air chamber of the pressure-receiver capsule 9. The air chamber is separated by a diaphragm from a line chamber, which is integrated or mounted in a line system 11, in particular a blood tube line, of the extracorporeal blood treatment machine 3.

During operation of the extracorporeal blood treatment machine 3, an internal line pressure is present in the line chamber of the pressure-receiver capsule 9, which is transmitted via the diaphragm to the air chamber. As a result, a fluid pressure signal is formed in the air chamber, which is applied directly to the pressure sensor 7 through the coupling portion 8 or through the rigid, bending resistant fluid line 10 and which is converted into an electrical signal by the pressure sensor 7. At least one cable 12 is connected to the pressure sensor 7 as an electric line for transmitting the pressure measurement signal. The cable runs through the tubular retainer 5 or the gooseneck and enters the housing 2 at a mounting site where the fastening device 4 of the retainer 5 is mounted on the housing 2. This means that the cable 12 runs through the tubular retainer 5 in such a way that it is completely protected from the environment between the pressure sensor 7 and the housing 2. As a result, the cable 12 cannot be damaged by environmental influences such as clumsy users, aggressive disinfectants, etc. At the same time, the adjustability of the pressure sensor 7 is ensured by the gooseneck-like configuration of the retainer 5. Inside the housing 2 of the extracorporeal blood treatment machine 3, the cable 12 is connected to a control unit or an electronic unit 13, which receives and processes the signal from the pressure sensor 7.

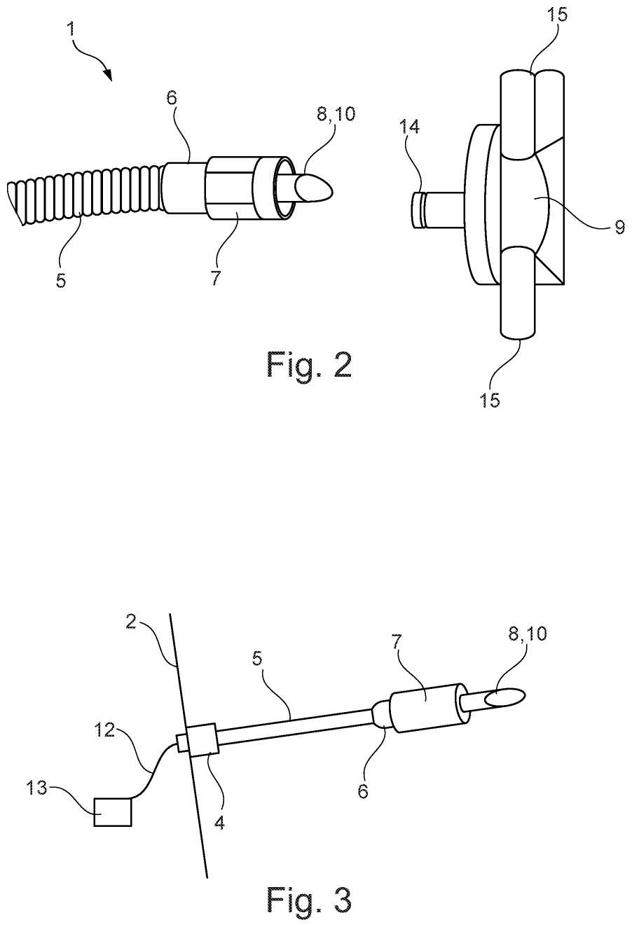

FIG. 2 shows a detailed view of the articulation site 6 of the retainer 5 holding the pressure sensor 7 during a coupling process with the pressure-receiver capsule 9. In this view, it is readily apparent that the coupling portion 8 of the pressure-measuring assembly 1, which is arranged on the pressure sensor 7, is formed as a male or female Luer-slip/lock connector 8 according to one aspect of the invention. Accordingly, the pressure-receiver capsule 9 has a counter coupling portion 14 in the form of a female or male Luer-slip/lock connector for coupling to the coupling portion 8 on one side of the air chamber. Furthermore, two line ports 15 fluidly connected to the line chamber of the pressure-receiver capsule 9 are provided, via which the pressure-receiver capsule 9 can be integrated or mounted in the blood tube line 11 of the extracorporeal blood treatment machine 3 in a fluid-conducting manner.

FIG. 3 schematically shows a pressure-measuring assembly 1 according to a second embodiment of the invention. This second embodiment corresponds in its basic structure to the first embodiment, which is why only their differences will be discussed below. According to the second embodiment, the retainer 5 has a rigid rod instead of a manually bendable gooseneck. In FIG. 3, the rod is shown running linearly as an example. Depending on the specific design of the extracorporeal blood treatment machine, in particular the arrangement of the various lines and housing feedthroughs, the retainer 5 or the rigid rod can also be curved and/or bent in order to achieve a course that is optimally adapted to the specific design.

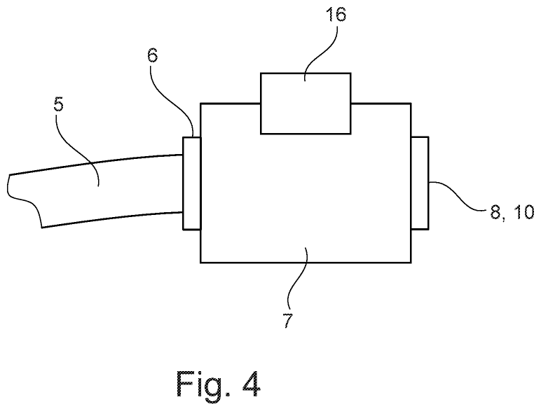

FIG. 4 schematically shows an articulation site 6 of a pressure-measuring assembly 1 according to the first or second embodiment to highlight further aspects of the invention. According to a first aspect, the pressure sensor 7 has as the coupling portion 8, instead of a Luer-slip/lock connector, an alternative connector such as a clickable or latchable connector element (shown here only schematically). According to a second aspect, which may be provided in addition to or as an alternative to the first aspect in either of the aforementioned embodiments, the pressure sensor 7 or the rigid, bending resistant fluid line 10 has a valve 16 integrated or mounted therein by which the air chamber of the pressure-receiver capsule 9 may be vented in order to move the diaphragm of the pressure-receiver capsule 9 to a particular position.

1. A pressure-measuring assembly for measuring the internal line pressure of a line system of an extracorporeal blood treatment machine, the pressure-measuring assembly comprising:
a pressure sensor which is connectable via a fluid line to a pressure receiver connected or connectable to the line system and which converts a fluid pressure signal from the fluid line into an electrical signal;
an electric line, which serves to connect the pressure sensor to electronics for processing the electric signal; and
a retainer for directly or indirectly holding the pressure sensor,
the fluid line being rigid and resistant to bending, and
the retainer forming, at least in sections, an inner channel in which the electric line is guided to protect the electric line from the environment.

2. The pressure-measuring assembly according to claim 1, wherein the retainer is formed as a plastically bendable rod having, at one axial end portion, a fastening device for being mounted to a stationary base and, at the other axial end portion, an articulation site for the pressure sensor or the pressure receiver.

3. The pressure-measuring assembly according to claim 1,
wherein the retainer holds the pressure sensor,
wherein the pressure sensor holds or is attached to the fluid line, and
wherein the fluid line is adapted to be attached to the pressure receiver.

4. The pressure-measuring assembly according to claim 1, wherein the retainer is attachable to a housing portion of the extracorporeal blood treatment machine.

5. The pressure-measuring assembly according to claim 1, wherein the fluid line is arranged on the pressure sensor and is rigid and resistant to bending throughout the fluid line.

6. The pressure-measuring assembly according to claim 1, wherein the inner channel forms a receiving space for receiving the electrical line that is completely closed with respect to the environment.

7. The pressure-measuring assembly according to claim 1, wherein the retainer is plastically deformable or curvable in such a way that a position of the articulation site is adjustable.

8. The pressure-measuring assembly according to claim 7, wherein the retainer is configured as a gooseneck which forms the inner channel.

9. The pressure-measuring assembly according to claim 1, wherein the retainer is a rod extending in a predetermined linear or curved shape.

10. The pressure-measuring assembly according to claim 1, wherein the pressure receiver is a pressure-receiver capsule comprising a first chamber connected or connectable to the line system and a second chamber separated from the first chamber by a diaphragm, the pressure sensor is being coupled or coupleable to the second chamber.

11. The pressure-measuring assembly according to claim 10, further comprising a valve integrated or mounted on the pressure sensor or on the fluid line to vent the pressure-receiver capsule.

12. The pressure-measuring assembly according to claim 1, wherein the pressure sensor has a Luer connection or a latchable connecting element as a coupling portion for directly coupling the pressure sensor to the pressure-receiver capsule.

13. An extracorporeal blood treatment machine comprising a pressure-measuring assembly according to claim 1.

14. The extracorporeal blood treatment machine according to claim 13, further comprising a housing portion, wherein the retainer is attached to a surface of the housing portion.

15. The extracorporeal blood treatment machine according to claim 14, wherein the surface of the housing portion faces an outside of the housing.

16. The pressure-measuring assembly according to claim 10, wherein the fluid line is a gas line and the second chamber is a gas-filled chamber.

17. The pressure-measuring assembly according to claim 11, wherein the valve is provided to vent the second chamber.
